(19) United States
(12) Patent Application Publication
(10) Pub. No.: US 2004/0077937 A1
(43) Pub. Date: Apr. 22, 2004

(54) Apparatus and method for coupling a medical device to a body surface

(75) Inventor: Orit Yarden, Givat Shmuel (IL)

Correspondence Address: Bingham McCutchen, LLP, Suite 1800, Three Embarcadero, San Francisco, CA, 94111-4067, US

(73) Assignee: Remon Medical Technologies Ltd

(21) Appl. No.: 10/277,337

(22) Filed: Oct. 21, 2002

(51) Int. Cl. A61B 5/04

(52) U.S. Cl. 600/386

(57) ABSTRACT

A device for pressing a medical operative element towards a body surface of a user is provided. The device includes a structure adapted for carrying the medical operative element, a restraining mechanism adapted to maintain the structure in a position, and a support. The support is secured to the structure at a position such that when a part of a body of the user is rested on the support, a force vector is created in the direction required to press the medical operative element towards the body surface of the user.

### FIELD OF THE INVENTION

The present invention is directed towards the field of body-contacting medical devices, and more particularly towards systems and methods for coupling a body-contacting medical device to a body surface.

### BACKGROUND OF THE INVENTION

Body-contacting medical devices, such as monitoring devices, and other therapeutic devices, which require a component of the device to contact a body surface of a user during use, are well known. Often, the preferable point of contact is on the torso (abdomen or thorax) of the user. The component of the device that contacts the body surface of the user is typically a sensor or therapeutic device, such as, e.g., a transducer, transponder, electrode, etc. Examples of clinical modalities that require use of a body-contacting device include Electrocardiogram (ECG), impedance cardiography, respiration monitoring, physiotherapy, thermometry, Doppler echocardiography, fetal monitoring, and electromyogram. Some of these applications may require several components of the device to be simultaneously placed in different locations on the body surface of the user.

In certain situations, i.e., in a home environment, it is desirable that the user be able to operate a body-contacting medical device on himself/herself without the assistance of others. However, several challenges exist. First, the users of home-monitoring devices and other similar devices are frequently elderly and/or disabled. These users may be too weak or may not have the physical capability to operate the device without the assistance of others.

Even if the users are not elderly and/or disabled, creating and maintaining body contacting force to press the body-contacting device onto the body surface of the user over a duration of period may be strenuous for the user. This is particularly the case if the device is hand-held. Also, the device may be too heavy to comfortably hold steady by hand for the duration of the operation of the device.

Also, the operation of the body-contacting device may require accurate positioning of the device in defined location(s) of the body surface. Sometimes the defined location may be difficult to reach by the user. Even if the defined location is reachable by the user, the user may not have the expertise and/or the coordination skills to accurately position the device.

Lastly, operation of the body-contacting device may require application of a coupling media, i.e., a gel, for improving an electrical or acoustical conductivity between the body-contacting device and a body surface of the user. The user of the body-contacting device may apply the coupling media inaccurately. For example, the user may apply too much or too little coupling media, which can negatively affect the performance of the device. Furthermore, if the user is a mentally disabled patient, or is otherwise absent-minded, there is a possibility that he/she may even forget to apply the coupling media.

For the foregoing, it is believed that an apparatus for coupling a body-contacting device to a body surface is needed that can address one or more of the above challenges.

### SUMMARY OF THE INVENTION

A device for pressing a medical operative element onto a body surface of a user is described. The device includes a structure adapted for carrying the medical operative element, a fastening mechanism adapted to maintain the structure in a position, and a support. The support is secured to the structure at a position such that when a part of a body of the user is rested on the support, a force vector is created in the direction required to press the medical operative element towards the body surface of the user.

Other embodiments of the device and methods of using the same are also described. Other and further aspects and features of the invention will be evident from reading the following detailed description of the preferred embodiments, which are intended to illustrate, not limit, the invention.

### BRIEF DESCRIPTION OF THE DRAWINGS

The drawings illustrate the design and utility of preferred embodiments of the present invention, in which similar elements are referred to by common reference numerals. In order to better appreciate how the above-recited and other advantages and objects of the present inventions are obtained, a more particular description of the present inventions briefly described above will be rendered by reference to specific embodiments thereof, which are illustrated in the accompanying drawings. Understanding that these drawings depict only typical embodiments of the invention and are not therefore to be considered limiting of its scope, the invention will be described and explained with additional specificity and detail through the use of the accompanying drawings in which:

FIG. 1 is a front view of a device in accordance with a first embodiment of the present invention;

FIG. 1B is a front view of a variation of the device of FIG. 1, particularly showing a part of the operative element being the structure;

FIG. 2 is a front view of the device of FIG. 1, particularly showing the device having a cable adapted to be connected to a feedback system;

FIG. 3A is an isometric view of the device of FIG. 1, particularly showing the wire grid on the back side of the structure;

FIG. 3B is an isometric view of the device of FIG. 1, particularly showing the structure having a hook and loop configuration(VELCRO) connection for detachably securing the operative element;

FIG. 3C is an isometric view of a variation of the device of FIG. 3A, particularly showing the operative element capable of being secured to the structure of the device at different positions;

FIGS. 4A-4D are side views of variations of the device of FIG. 1, particularly showing variations of the shoulder strap(s);

FIG. 4E is an isometric view of a variation of the device of FIG. 1, particularly showing two shoulder straps and a chest strap;

FIGS. 5A and 5B are front views of variations of the device of FIG. 1, particularly showing the length of the shoulder strap being adjustable;

FIG. 6 is a side view of the device of FIG. 1, particularly showing a user using the device while lying down;

FIG. 7 is an isometric view of a variation of the device of FIG. 1, particularly showing the handle being adjustable;

FIG. 8 is a side view of the device of FIG. 7, particularly showing a user using the device while standing straight up;

FIG. 9A is a front view of a variation of the device of FIG. 1, particularly showing the handle being slidable;

FIG. 9B is a front view of a variation of the device of FIG. 1, particularly showing the handle being extendable;

FIG. 10 is a front view of a variation of the device of FIG. 1, particularly showing a handle having two branches;

FIG. 11A is a front view of a variation of the device of FIG. 1, particularly showing the device having armrest(s) as the support;

FIG. 11B is an end view of the platform of the armrest of FIG. 11A;

FIG. 12A is a front view of a variation of the device of FIG. 11A, particularly showing the position of the platform being adjustable;

FIG. 12B is a top view of another variation of the device of FIG. 11A, particularly showing the position of the platform being adjustable;

FIG. 13A is a front view of a variation of the device of FIG. 11A, particularly showing the member connected to a block that is slidable relative to the structure of the device;

FIG. 13B is a side view of a variation of the device of FIG. 13A, particularly showing the block having a first portion that is rotatable relative to a second portion;

FIG. 14A is a partial front view of a member of the device, particularly showing the platform being adjustable in three degrees of freedom;

FIG. 14B is a partial front view of a member of the device, particularly showing the member being extendable; and

FIG. 15 is a partial side cross-sectional view of a structure of the device in accordance with a second aspect of the present invention, particularly showing the device having an automatic dispensing unit.

### DETAILED DESCRIPTION OF THE PREFERRED EMBODIMENTS

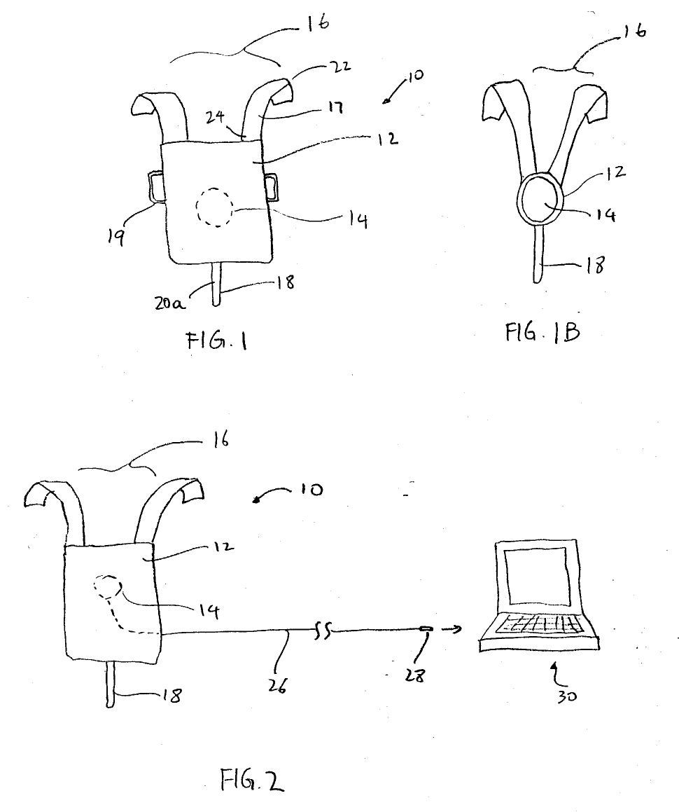

FIG. 1 is a front view of a device 10 in accordance with a first preferred embodiment of the present invention. The device 10 includes a structure 12 for carrying an operative element 14, a restraining mechanism 16 for immobilizing the structure 10 and operative element 14, and a support 18 that can be manipulated by the user to press the operative element 14 towards the user. Although not required, the device 10 may further include one or more transport handles 19, which a user can use to lift or carry the device 10.

The structure 12 of the device can be made of a variety of materials, such as plastics, light weight metals, fabrics, styrofoam, leather, nylons, and combinations thereof. Depending on the particular application, the structure 12 may include a reinforcing material, such as a wire mesh or a light gage metal plate, to improve the strength and/or stiffness of the structure 12. Although FIG. 1 shows that the structure 12 has a general shape of a rectangle, the structure 12 can have other shapes and dimensions as well. Although FIG. 1 shows the operative element 14 as being separate from the structure 12, the operative element 14 can be integral with the structure 12 or form the structure 12 itself. For example, FIG. 1B shows a variation of the device of FIG. 1, particularly showing a part of the operative element 14 forming the structure 12. In particular, a casing of the operative element 14 is being used as the structure 12. As such, the support 18 and the restraining mechanism 16 are secured to the casing of the operative element 14.

The operative element 14 is preferably a medical monitoring device that senses energy or a signal (such as electrical signal, acoustic signal, and thermal signal) from a user. For examples, the operative element 14 can be a thermistor, a respiration sensor, a fetal monitoring device, a cardiac signal sensor, and monitoring device for use in impedance cardiography, physiotherapy, thermometry, and Doppler echocardiography. Alternatively, the operative element 14 can also be a therapeutic device that sends energy (such as thermal energy, electrical energy, and acoustic energy) to the contacting surface of the body of a user, or a drug delivery device that delivers medication to the user through the contacting surface of the body of the user. The operative element 14 can also transmit and receive signals and/or data. For example, the operative element 14 can transmit and/or receive acoustic energy to and from an implant to measure a characteristic of a patient, such as pressure. The operative element 14 can also transmit energy or signals to energize an implant, or send data to an implant to program the implant. Furthermore, the operative element 14 is not limited to devices that require direct contact with a body surface of a user during an operation. For example, the operative element 14 also includes devices that may be placed directly against a clothing of a user during an operation.

Referring to FIG. 2. In one embodiment, the device 10 optionally includes a cable 26 coupled to the operative element 14, with one end 28 of the cable 26 being adapted to connect to a feedback device 30, such as a monitor, a printer, or a computer, for generating feedback to a user based on an operation of the operative element 14. Alternatively, the device 10 can include a radio frequency transmitter for wirelessly communicating a signal between the operative element 14 and a computer or the like. The signal can be a digital signal or an analog signal. As a further alternative, the device 10 can include a communication device, such as a phone port, a modem, or a cable port, through which information associated with an operation of the operative element 14 can be transmitted. As another alternative, the device 10 can include a screen for displaying information to a user. The screen can be located on the structure 12 or on the operative element 14. The screen can also be a part of a separate hand-held unit directly or wirelessly coupled to the structure 12 or the operative element 14.

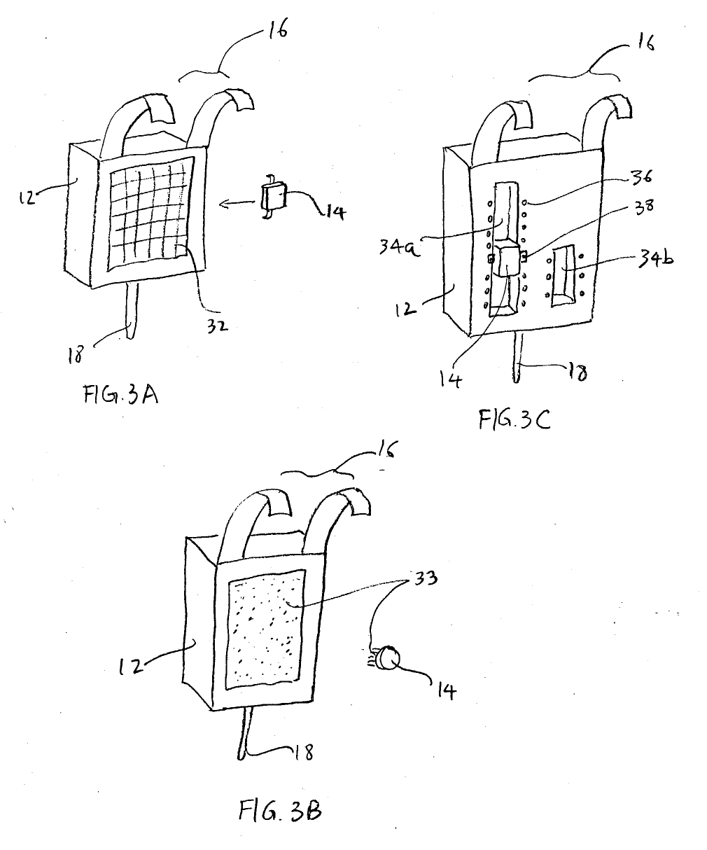

Preferably, the operative element 14 is capable of being secured to different positions on the structure 12 of the device 10. For example, as shown in FIG. 3A, the structure 12 can include a wire grid 32 to which the operative element 14 can be secured at various positions. Alternatively, as shown in FIG. 3B, the operative element 14 can be secured to the structure 12 of the device 10 at various positions using a VELCRO connection 33. FIG. 3C shows that the structure 12 can also include one or more pre-formed openings 34 and/or indentations with various shapes and sizes in which the operative element 14 can be placed and be secured at different positions. The operative element 14 is slidable in the opening 34a, and can be secured at a position by placing screws into one of the pairs of opposing flanges 38 integrally formed with the operative element 14 and then through one of the pairs of opposing openings 36 straddling the opening 34a on the structure 12. The operative element 14 can also be placed and secured in the second opening 34b if it is desired. In yet another embodiment, the device 10 may include a notched bar along which the operative element 14 can be moved and locked in any one of the notches.

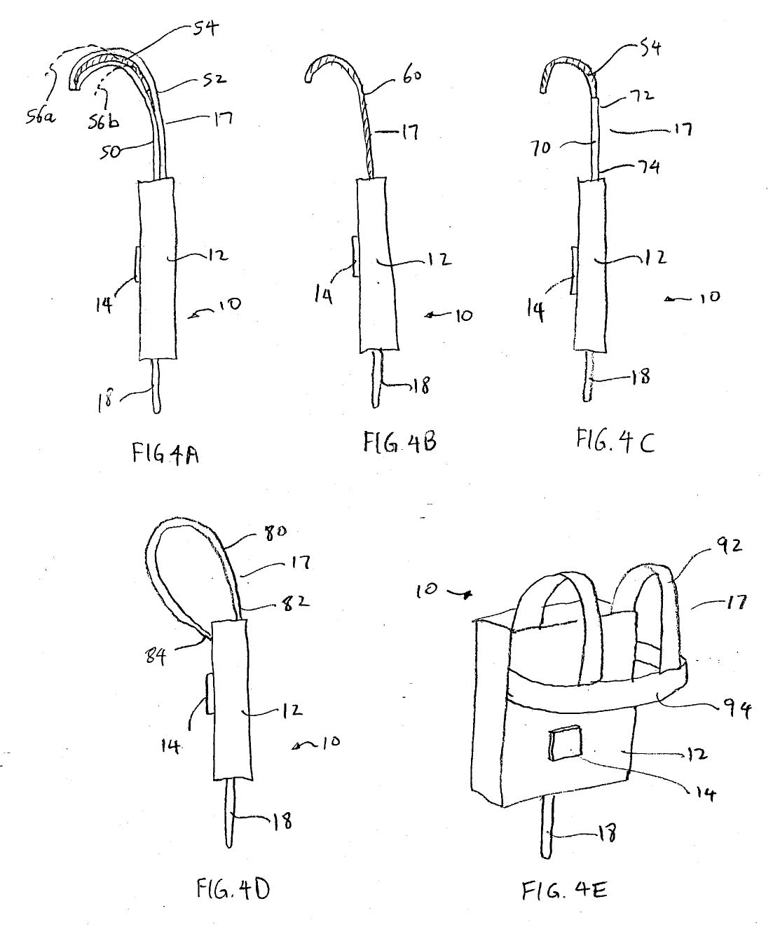

Alternatively, if it is not necessary to provide multiple securing positions for the operative element 14, the operative element 14 can be secured to one predetermined position on the structure 12 by a connection, such as a screw or an adhesive. The operative element 14 may be permanently or detachably secured to the structure 12 during a manufacturing process or before an operation of the device 10. It should be noted that for any of the examples discussed previously, a portion or all of the operative element 14 can either protrude above a surface of the structure 12, such as that shown in FIG. 4A, or be flush with the surface of the structure 12.

The operative element 14 is preferably activated (i.e., turned on) and/or operated (i.e., perform sensing, monitoring, etc.) by a remote control, which could be provided as a part of the device 10. The remote control can also be used to activate a computer or a feedback system (if one is provided). Alternatively, the device 10 can include one or more control buttons located on the structure 12 or on the operative element 14 for control of the operative element 14. Methods of activating and/or operating the operative element 14 will be described below.

In general, the restraining mechanism 16 is for securing the structure 12 (and therefore, the operative element 14) in a position relative to a body surface of the user. In the example shown in FIG. 1, the restraining mechanism 16 comprises one or more shoulder straps 17, each of which has a top end 22 adapted to secure to a shoulder of a user, and a bottom end 24 affixed to the structure 12.

The shoulder strap 17 is preferably made of an elastic material, such as aluminum, so that the top end 22 can be bent into a desired shape to fit a shape and size of a particular user's shoulder. FIG. 4A shows an embodiment of the shoulder strap 17 that includes a bottom cover 50, a top cover 52, and a bendable metal plate 54 disposed between the bottom cover 50 and the top cover 52. The metal plate 54 is located at the top end 22 of the strap 17, and is preferably secured to the bottom cover 50 and top cover 52 by an adhesive, screw(s), or bolt(s). Dash-lines 56 represent various shapes in which the top end 22 of the shoulder strap 17 can assume by bending the metal plate 54 within the strap 17. FIG. 4B shows an alternative embodiment of the strap 17, for which the entire length of the strap 17 is made of an elastic material 60, such as a metal plate. FIG. 4C shows another embodiment of the strap 17 that includes a metal plate 54 secured to a band 70 having a top end 72 and a bottom end 74. In particular, the metal plate 54 is secured to the top end 72 of the band 70, while the bottom end 74 of the band 70 is secured to the structure 12. The securing between the various components can be carried out using screw(s), bolt(s), and/or a suitable adhesive, as is known to those skilled in the art.

Alternatively, the shoulder strap 17 may not include the bendable elastic material as described previously. FIG. 4D shows a shoulder strap 17 that includes a shoulder band 80 having a first end 82 and a second end 84. The first end 82 and the second end 84 of the shoulder band 80 are secured to the structure 12, thereby forming the shoulder band 80 into a loop. FIG. 4E shows another variation of the shoulder strap 17 that includes a shoulder band 92 and a back strap 94. The shoulder band 92 is adapted to be placed around a user's shoulder, while the back strap 94 is adapted to wrap around a user's chest. As such, both the shoulder band 92 and the back strap 94 operate together to secure the structure 12 (and therefore, the operative element 14) relative to a body surface.

It should be noted that all the embodiments of the shoulder strap 17 described previously can be made adjustable in dimensions, locations, and/or orientations so that it can be used by users having different body shapes and sizes. For example, FIGS. 5A and 5B are front views of variations of the device 10, particularly showing the shoulder straps 17 being adjustable in length. FIG. 5A shows a variation of the shoulder strap 16, which includes a bottom portion 100 and a top portion 102. The top portion 102 of the strap 17 is configured for hanging onto a shoulder of a person. The bottom portion 100 is secured to the structure 12. Specifically, the bottom portion 100 includes a slot 104, and the top portion 102 includes a threaded pin 36 adapted to slide within the slot 104 of the bottom portion 100, thereby adjusting the length of the strap 17. When the threaded pin 106 is positioned at a desired location relative to the slot 104, a screw cap (not shown) is then screwed onto the threaded pin 106, thereby securing the top portion 102 of the strap 16 relative to the bottom portion 100 of the strap 17.

FIG. 5B shows another variation of the shoulder strap 17, which includes a plurality of adjustment openings 112, a bottom end 114, and a securing mechanism 116 secured at the bottom end 114 of the shoulder strap 17. During use of the device 10, the bottom end 114 of the strap 17 is placed through an opening 118 on the structure 12, and the securing mechanism 116 is then mated with one of the adjustment openings 112 in order to secure the strap 17, having a desired length, to the structure 12. Alternatively, the securing mechanism 116 can be a belt buckle or other similar devices. It should be noted that the types of adjustable straps are not necessarily limited to the examples described above, and that other types of adjustable straps known in the art may also be used.

Although several examples of the restraining mechanism 16 in a form of a shoulder strap have been described, it should be understood that the device 10 can include other types of restraining mechanisms. For examples, the restraining mechanism 16 can be a chest strap that is adapted to wrap around a chest of the user. The restraining mechanism 16 can also be neck strap, an arm strap, a leg strap, or other kinds of strap for strapping, either partially or completely, around a body part of a user. Furthermore, the restraining mechanism 16 can be a rope, a cable, a chain, a hoist, a sling, and other forms of restraint known in the art, so long as the restraining mechanism 16 can assist immobilization of the structure 12 (and therefore, the medical operative element 14) relative to a body surface of the user.

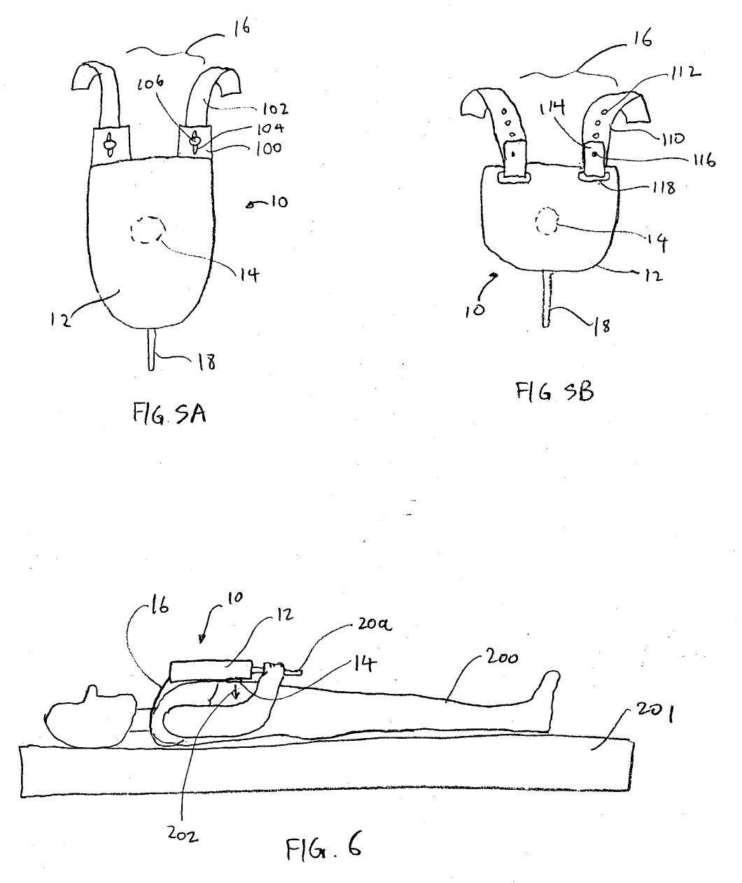

Returning to FIG. 1, the support 18 is designed so that when a user's arm or hand rests on the support 18, the weight of the arms and/or hands of a user applies a force in the direction required to press the operative element 14 towards the surface of a user's body for creation of adequate coupling between the operative element 14 and the body surface of the user. As the example shown in FIG. 1, the support 18 is a handle 20a that is secured to a bottom of the structure 12. The handle 20a is for a user to rest his/her hand(s). FIG. 6 shows a side view of the device 10 of FIG. 1 being used by a user 200 who is lying on a bed 201. When the user 200 rests his/her hand(s) on the handle 20a, the weight of the hand(s) and/or the arm(s) create a force in a direction, as represented by arrow 202, that is required to press the operative element 14 towards the body surface of the user 200.

The support 18 is preferably adjustable so that a user can press the medical operative element 14 towards a body surface in a variety of positions. FIG. 7 shows a variation of the device 10 that includes an adjustable handle 20b as the support 18. In particular, the handle 20b includes a hinge 220 that allows a bottom end 222 of the handle 20b to rotate in a variety of angles 224. A tightening knob (not shown) can be used to secure the bottom end 222 of the handle 20b when a desired angle 224 is obtained. Alternatively, the bottom end 222 of the handle 20b can also be secured by a friction-type connection or other securing mechanisms known in the art.

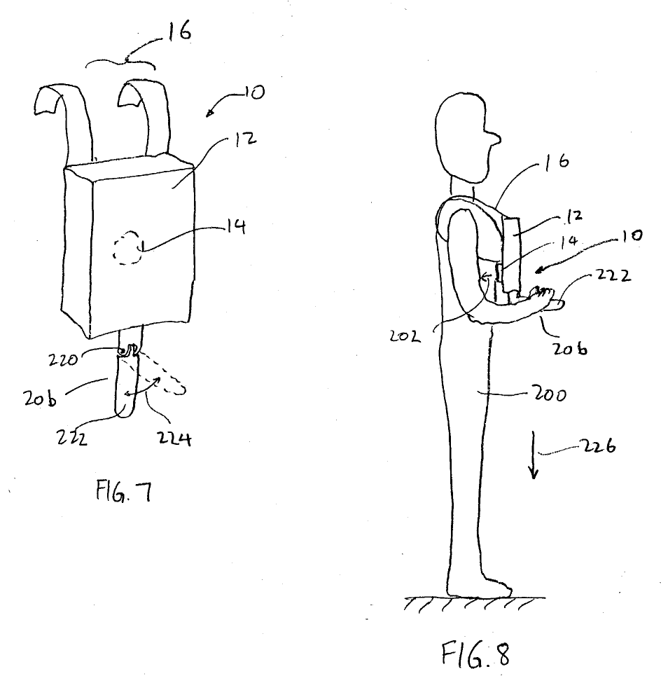

FIG. 8 shows a side view of the device 10 of FIG. 7 being used by a user 200 who is standing straight up. In particular, the angle 224 of the handle 20b has been adjusted so that the bottom end 222 of the handle 20b is substantially perpendicular to an acceleration vector 226 of the gravity. When the user 200 rests his/her hand(s) on the handle 20b, the weight of the hand(s) and/or the arm(s) create a force in a direction, as represented by arrow 202, that is required to press the operative element 14 towards the body surface of the user 200. As such, by adjusting the angle 224 of the handle 20b, the operative element 14 can be activated by the user in various positions, i.e., lying flat, inclined, declined, sitting down, and standing straight up.

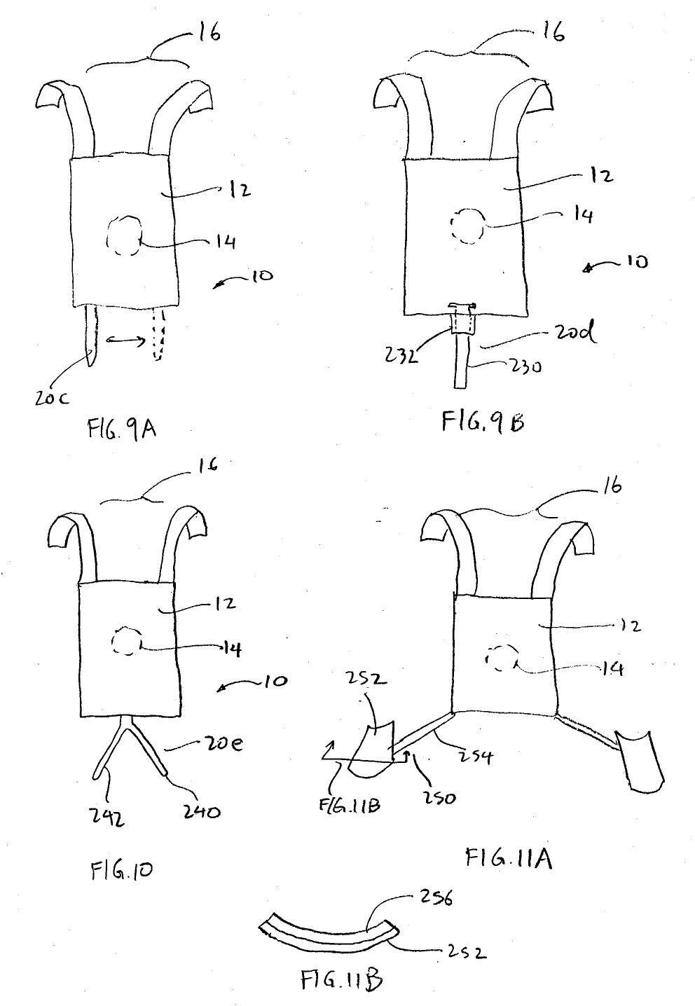

The handle 20 can also be adjustable in other ways. FIG. 9A shows a handle 20c that is adjustable within a plane of the structure 12. In particular, the handle 20c is slidably secured to the structure 12 so that a user may adjust the position of the handle 20c by sliding the handle 20c, i.e., within a grove or slot (not shown) on the structure 12. FIG. 9B shows a handle 20dthan can be extended in its length. The handle 20d includes an elongate member 230 that is coaxially located within a tubular element 232. The tubular element 232 of the handle 20d can be positioned relative to the elongate member 230, thereby, changing the length of the handle 20d. Such feature allows users having different hand size to use the device 10. Also, such feature is advantageous in that a user can extend the handle 20d if he/she desires to rest two hands onto the handle 20d, and can retract the handle 20d if he/she desires to rest only one hand onto the handle 20d. It should be understood that the handle 20 is not limited to those described previously, and that the position and orientation of the handle 20 can be made adjustable using a variety of techniques and devices known in the art.

It should also be noted that the support 18 is not necessarily limited to the handles 20 described previously, and that it can have other configurations as well. For example, FIG. 10 shows a handle 20e, which includes a left branch 240 and a right branch 242. The left branch 240 is for a user's left hand to rest on, while the right branch 242 is for a user's right hand to rest on. The user may rest one hand or both hands on the handle 20e during use of the device 10. As discussed previously, either or both of the branches 240 and 242 of the handle 20e can be made adjustable in a variety of positions.

FIG. 11A shows another variation of the support 18, which includes a pair of armrests 250. In particular, each armrest 250 includes a platform 252 on which a user's arm can rest. The platform 252 is secured to the structure 12 of the device 10 through a strut 254. Although FIG. 11A shows that the device 10 has two armrests 250, it should be understood that the device 10 can have only one armrest 250 as well.

As shown in FIG. 11B, the cross section of the platform 252 preferably has a curvilinear shape so that the user's arm can be prevented from shifting away from the armrest 250 during use of the device 10. Alternatively, the cross section of the platform 252 can have a “V” shape, a linear (flat) shape, or other customized shape. The platform 252 is preferably made of an elastic metal that is bendable so that the cross sectional shape of the platform 252 can be modified by a user. The platform 252 can also be made of plastics, wood, carbon-fiber, and other materials known to those skilled in the art. Although not required, the armrest 250 can further include a support layer 256 secured to a surface of the platform 252. The support layer 256 provides a more comfortable surface for the user's arm to rest on. The support layer 256 can be made of a variety of material, such as polyester, cotton, etc.

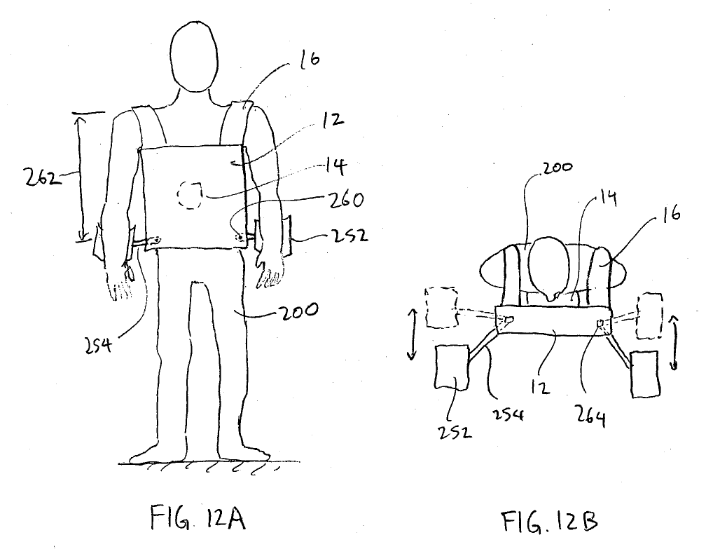

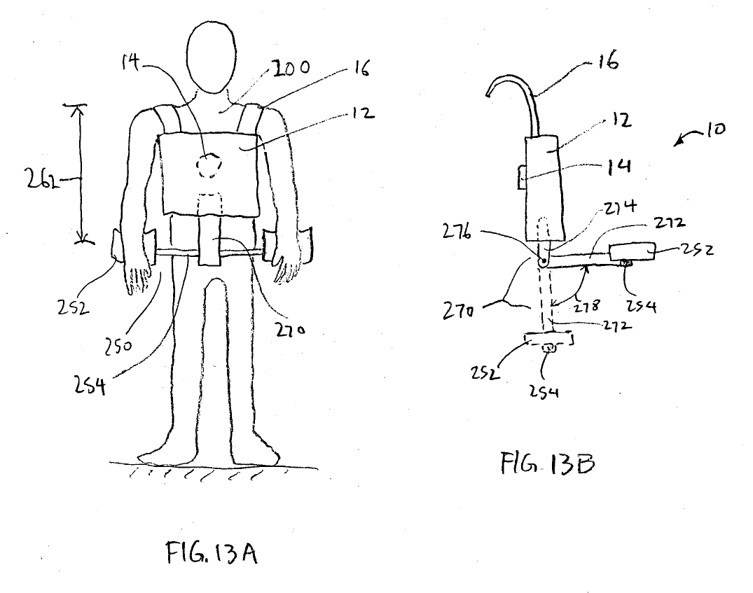

The struts 254 can be made of a variety of materials, such as metals, plastics, alloys, and timbers, so long as it can transfer forces from the platform 252 to the structure 12 of the device. The strut 254 is preferably moveable in at least one degree of freedom so that the platform 252 can be adjusted in position relative to the structure 12. FIGS. 12-14 show examples of armrests 250 that are adjustable relative to the structure 12.

FIG. 12A shows a platform 252 of an armrest 250 connected to the structure 12 by the strut 254, which is rotatable relative to the structure 12. In particular, one end of the strut 254 is rotatably secured to the structure 12 by a hinge 260. Such configuration allows a user to adjust a vertical distance 262 between the platform 252 and the user's shoulder. FIG. 12B is a top view of another example of the armrest 250 in which the strut 254 is rotatably secured to the structure 12 by a hinge 264. The hinge 264 allows the user to rotate the strut 254 of the armrest 250 along an axis that is different from that shown in FIG. 12A.

FIG. 13A shows another variation of the armrest 250 in which the strut 254 is slidably connected to the structure 12. The strut 254 is connected to a flange 270 that is adapted to slide relative to the structure 12. By sliding the flange 270 in and out of the structure 12, a user can adjust the vertical distance 262 between the platform 252 and the user's shoulder. FIG. 13B shows a variation of the armrest 250 for which the extendable flange 270 includes a first portion 272 and a second portion 274. In particular, the first portion 272 is rotatably secured to the second portion 274 by a hinge 276, such that the first portion 272 can be rotated at different angle 278 relative to the second portion 274. As similarly described with reference to FIGS. 7 and 8, by adjusting the angle 278 between the first portion 272 and the second portion 274 of the armrest 250, the operative element 14 can be pressed towards a body surface of a user in various positions.

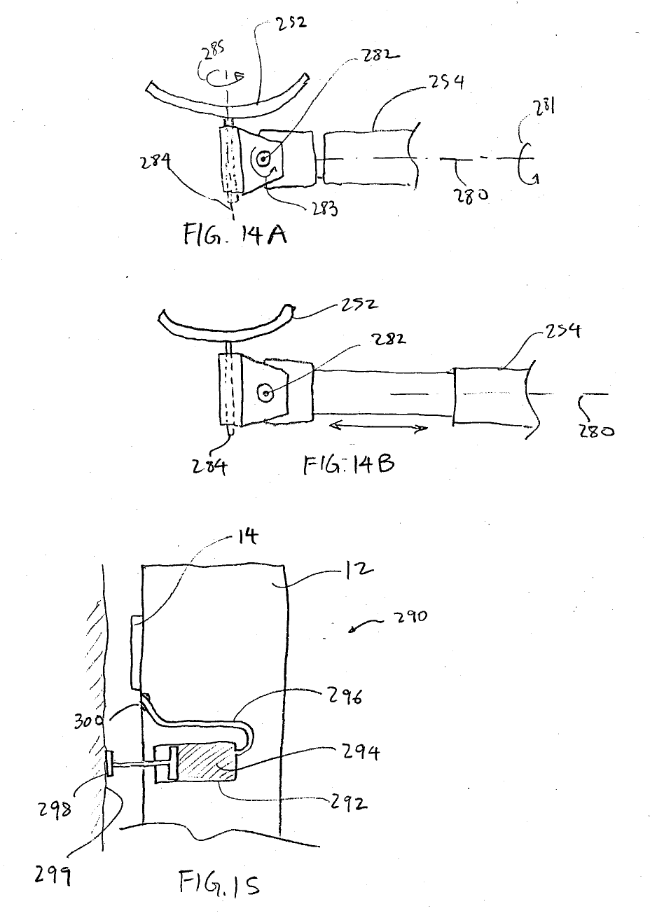

Although not required, the platform 252 of the armrest 250 can also be made adjustable relative to the strut 254 in one or more degrees of freedom. FIG. 14 shows an example of the platform 252 that is rotatable relative to the strut 254 in three degrees of freedom. The platform 252 is rotatable about orthogonal axes 280, 282, and 284. Specifically, the platform 252 may rotate about axis 280, as indicated by arrow 281, axis 282, as indicated by arrow 283, and axis 284, as indicated by arrow 285. optionally, the strut 254 can further be made extendable along axis 280, as illustrated in FIG. 14B.

It should be noted that the adjustability of the armrest 250 can be implemented in a variety of ways, and that the foregoing are only examples of how the armrest 250 can be made adjustable. Any or all of adjustable features described previously can be implemented using hinge connections (which can be set and tightened to a specific position), shaft connections (for coupling elongate members), free hinges (which will self-set according to gravitational force) and/or other types of connection known to those skilled in the art.

Although the embodiments of the support 18 shown in FIGS. 11-14 were described with reference to an armrest, it should be understood that the platform 252 of the armrest 250 can also be used to support other body part(s), such as a hand, of a user. In addition, the strut 254 can be used to connect the platform 252 to other parts of the structure 12. Furthermore, the strut 254 of the armrest 250 is not necessarily limited to an elongate shaft like that shown in the previous examples. The strut 254 can have a variety of forms, shapes, and configurations, so long as the strut 254 can transfer a force from the platform 252 to the structure 12. For examples, the strut 254 can have a different length and cross-sectional shape, can be in a form of a plate, and can be a variety of devices that function as a connection connecting the platform 252 to the structure 12.

For any of the above described embodiments, the operative element 14 can be activated and/or operated in a variety of ways. In one embodiment, the activation and/or the operation of the operative element 14 are performed when the user 200 rests or places a part of his/her body on the support 18. For example, the device 10 can include a sensor located on the support 18 or structure 12, which is coupled to the operative element 14. When the sensor senses a characteristic, such as a pressure or a temperature, of the user 200, the operative element 14 is then activated and/or set to perform its function(s) Alternatively, instead of using a sensor, the support 18 can be mechanically coupled to the operative element 14, such that the resting of a part of a user's body on the support 18 will cause the operative element 14 to activate and/or to operate.

In another embodiment, a remote control is provided, and the operation of the operative element 14 is performed when the user pushes a button on the remote control. Such can be advantageous especially when the user rests his/her arm or forearm on the support 18, in which case the user can still use his/her hand(s) to operate the remote control. Alternatively, the device 10 can include one or more buttons conveniently located on the structure 12 or on the operative element 14 such that the user 200 can activate and/or operate the operative element 14 with his/her hand(s) even when his/her arm(s) or forearm(s) is rested on the support 18.

It should be noted that the scope of the present invention should not be limited by how the operative element 14 is activated. In fact, the operative element 14 can be independently activated and set to perform operation(s) without the help of the device 10. The device 10 is then used to create the required coupling between the operative element 14 and the body surface of the user.

Certain types of the operative element 14 may require a coupling media to be applied between the body surface of the user and the operative element 14. If a coupling media is required, it can be applied manually onto a surface of the operative element 14 or a body surface of the user before the operative element 14 is activated or operated. Alternatively, the device 10 can include a dispensing unit for dispensing coupling media. FIG. 16 shows a side view of the structure 12 of the device 10 that includes an automatic dispensing unit 290. The automatic dispensing unit 290 includes a compartment 292 for storing a coupling media 294, a tube 296 in fluid communication with an interior of the compartment 292, and a plunger 298. During use, the resting or placing of a part of a user's body on the support 18 causes the structure 12 to be pressed towards a body surface 299, as described previously. When this happens, the body surface 299 pushes the plunger 298 into the compartment 292 of the automatic dispensing unit 290, thereby, causing a pressure within the compartment 292. Due to the internal pressure within the compartment 292, the coupling media 294 is then dispensed into the tube 296, which delivers the coupling media 294 to a target area through an opening 300 on or adjacent to the operative element 14. The automatic dispensing unit 290 may further include a flexible valve (not shown) at the opening 300, which opens automatically when the coupling media 294 passes through the opening 300.

It should be noted that the automatic dispensing unit 290 is not limited to the example discussed previously, and that automatic dispensing units having different configurations may also be used. For example, the plunger 298 of the dispensing unit 290 may be mechanically coupled to the support 18 of the device 10, such that the resting of a user's arm on the support 18 presses the plunger 298 into the compartment 294. Alternatively, the dispensing unit 290 can include a hand-activated button located on the structure 12 or on the operative element 14, the pressing of which causes the coupling media 294 to be dispensed. Hand-activated media dispensing devices are well known in the art. As yet another alternative, the automatic dispensing unit 290 may include a sensor for sensing a characteristic, such as a temperature or a pressure, of a user. The sensor may be placed on the support 18 or on the structure 12. During use, when the sensor senses a characteristic of a user, the sensor transmits an electrical signal to an electrically-activated pump located on the structure 12, which then causes the coupling media 294 to be dispensed. Alternatively, instead of using a pump, an electrically-activated motor can be used to open a mechanical valve for dispensing the coupling media 294. Electrically-activated pumps and motors are well known in the art.

Having described the structure of the device 10, its operation will now be described. When using the device 10, a user initially places the restraining mechanism 16 partially or completely around a part of his/her body so that the structure 12 is secured at a position relative to a body surface of the user. If the restraining mechanism 16 is adjustable, the user can adjust a length, a position, or an orientation of the restraining mechanism 16 so that the structure 12 (and therefore, the operative element 14) can be secured at a desired position. If the positioning of the operative element 14 relative to the structure 12 is adjustable, the user can also change the position of the operative element 14 relative to the structure 12 by securing the operative element 14 to a desired position on the structure 12, as described previously. The adjustment of the position of the operative element 14 may be performed by the user, a care taker, or a professional care provider, either periodically, or in a one-time setup.

Next, depending on the position of the user in which the user wishes to operate the operative element 14, the position of the support 18 is adjusted. In particular, the position of the support 18 is adjusted such that when the user rests or places a part of his/her body (i.e., an arm, a forearm, or a hand) on the support 18, a force is created in the direction that is required to activate and/or operate the operative element 14, as described previously.

Once the support 18 is adjusted to a desired position, the user then rests or places a part of his/her body on the support 18 to create a force in a direction required to press the operative element 14 towards a body surface of the user. The created force should be sufficient such that the operative element 14 maintains an adequate coupling (i.e., substantial contact) with the body surface of the user. If the operative element 14 is placed against a clothing that is in direct contact with the body surface of the user, the coupling with the body surface of the user can be created by maintaining the operative element 14 in substantial contact with the clothing. If a desired coupling between the operative element 14 and the body surface of the user cannot be created, the location of the support 18 can be further adjusted until the support 18 is in a position such that a required force for creating adequate coupling can be created by resting or placing a part of a user's body on the support 18.

If the device 10 is designed such that the operative element 14 activates and/or operates dependently of the support 18, the operative element 14 is then automatically activated and/or operated by virtue of the user resting his body part, on the support 18. If the device 10 is designed such that the operative element 14 is operated independent of the support 18, the user will manually activate the operative element 14, and then create a desired coupling between the operative element 14 and a body surface by resting his body part on the support 18.

Thus, although several preferred embodiments have been shown and described, it would be apparent to those skilled in the art that many changes and modifications may be made thereunto. For example, the structure 12 may be strapped around a body of a user and may include an inflatable bladder. An inflation of the bladder, either by a user or a care taker, would increase the strapping force around the body, thereby improving the coupling between the operative element and the body surface of, the user. Other changes and modifications may also be made, without the departing from the scope of the invention, which is defined by the following claims and their equivalents.

What is claimed:

1. A device for pressing a medical operative element onto a body surface of a user, the device comprising:
a structure adapted for carrying the medical operative element;
a restraining mechanism secured to the structure, the restraining mechanism adapted to substantially immobilize the structure relative to the user; and
a support secured to the structure at a position such that when a part of a body of the user is rested on the support, a force is created in the direction required to press the medical operative element towards the body surface of the user.

2. The device of claim 1, wherein the structure comprises a wire grid to which the medical operative element can be secured at different positions.

3. The device of claim 1, wherein the operative element is detachably secured to the structure by a VELCRO connection.

4. The device of claim 1, further comprising a notched bar having one or more notches, the operative element configured to slide along the notched bar and is capable of being secured in a position by engaging the one or more notches.

5. The device of claim 1, further comprising the operative element secured to the structure.

6. The device of claim 5, wherein the medical operative element is a sensor.

7. The device of claim 6, wherein the sensor is a thermal sensor.

8. The device of claim 6, wherein the sensor is a cardiac signal sensor.

9. The device of claim 5, wherein the medical operative element is a therapeutic device.

10. The device of claim 9, wherein the therapeutic device delivers heat energy to the user.

11. The device of claim 9, wherein the therapeutic device delivers mechanical energy to the user.

12. The device of claim 5, wherein the medical operative element is adapted to transmit a signal to a receiver.

13. The device of claim 12, wherein the receiver is coupled to an implant.

14. The device of claim 12, further comprising a wire coupled to the operative element, the wire configured for transmitting information from the operative element to the receiver.

15. The device of claim 12, further comprising a wireless transmitter coupled to the operative element, the wireless transmitter configured for wirelessly transmitting information from the operative element to the receiver.

16. The device of claim 5, wherein the medical operative element is adapted to receive a signal from an implant.

17. The device of claim 5, wherein the medical operative element is adapted to send data to an implant.

18. The device of claim 5, wherein the medical operative element is adapted to energize an implant.

19. The device of claim 5, wherein the medical operative element is adapted to transmit and receive acoustic energy to and from an implant.

20. The device of claim 1, wherein the restraining mechanism is adjustable to accommodate a positioning of the medical operative element.

21. The device of claim 1, wherein the restraining mechanism comprises one or more shoulder straps.

22. The device of claim 1, wherein the restraining mechanism comprises a chest strap.

23. The device of claim 1, wherein the restraining mechanism comprises a neck strap.

24. The device of claim 1, wherein the support comprises one or more handles.

25. The device of claim 1, wherein the support comprises one or more armrests.

26. The device of claim 1, wherein the support is adjustable in position relative to the structure.

27. The device of claim 1, further comprising a transport handle secured to the structure.

28. The device of claim 1, further comprising a dispensing unit for dispensing a coupling media to the body surface.

29. The device of claim 28, wherein the dispensing unit comprising a compartment defining an interior for storing the coupling media, and one or more lumens that are in fluid communication with the interior of the compartment.

30. The device of claim 29, wherein the dispensing unit further comprises a sensor, and the dispensing unit is adapted to automatically dispense the coupling fluid when the sensor senses a characteristic from the user.

31. The device of claim 29, wherein the dispensing unit is mechanically coupled to the support, and the dispensing unit is adapted to automatically dispense the coupling fluid when the user rests a part of his/her body on the support.

32. A method of pressing a medical operative element onto a body surface of a user using a device having a structure, a restraining mechanism, and a support, the method comprising:
securing the medical operative element to the structure;
placing the restraining mechanism at least partially around a body part of a user to secure the structure relative to the body surface of the user; and
resting a part of the body on the support to create a force in the direction required to press the operative element towards the body surface of the user.

33. The method of claim 32, wherein the medical operative element securing is performed when the device is manufactured.

34. The method of claim 32, wherein the medical operative element securing comprises attaching the medical operative element to a wire grid of the structure.

35. The method of claim 32, wherein the medical operative element securing comprises attaching the medical operative element to the structure using a VELCRO connection.

36. The method of claim 32, wherein the restraining mechanism placement comprises placing the restraining mechanism at least partially around a neck of the user.

36. The method of claim 32, further comprising applying a coupling media between the medical operative element and the body surface.

37. The method of claim 32, wherein the restraining mechanism placement comprises placing the restraining mechanism at least partially around a chest of the user.

37. The method of claim 36, wherein the coupling media application is performed before the force is created.

38. The method of claim 32, wherein the restraining mechanism placement comprises placing the restraining mechanism at least partially around a shoulder of the user.

38. The method of claim 36, wherein the coupling media application is performed automatically when the force is created.

39. The method of claim 32, wherein the body part resting comprises placing an arm of the user on the support.

40. The method of claim 32, wherein the body part resting comprises placing a forearm of the user on the support.

41. The method of claim 32, wherein the body part resting comprises placing a hand of the user on the support.
